(19) United States
(12) Patent Application Publication
(10) Pub. No.: US 2004/0193216 A1
(43) Pub. Date: Sep. 30, 2004

(54) Incision closure device

(76) Inventor: Michael J. Deutsch, Burlington, WI (US)

Correspondence Address: QUARLES & BRADY LLP, 411 E. WISCONSIN AVENUE, SUITE 2040, MILWAUKEE, WI, 53202-4497, US

(21) Appl. No.: 10/782,713

(22) Filed: Feb. 19, 2004

Related U.S. Application Data
(60) Provisional application No. 60/448,686, filed on Feb. 19, 2003.

(51) Int. Cl. A61B 17/08

(52) U.S. Cl. 606/216; 606/213

(57) ABSTRACT

A kit provides a sterile set of hook and loop fastener elements two of which may be attached to sides of an incision to be joined across the incision with mating spanning elements to stepwise draw the incision without undue pressure or force concentration across the suture line.

### CROSS-REFERENCE TO RELATED APPLICATIONS

This application claims the benefit of U.S. Provisional application 60/448,686 filed Feb. 19, 2003, which is hereby incorporated by reference.

### STATEMENT REGARDING FEDERALLY SPONSORED RESEARCH OR DEVELOPMENT

### BACKGROUND OF THE INVENTION

The present invention relates generally to skin incision closure devices and in particular, to a device providing improved control of approximation and skin stretching forces between sides of an incision or skin opening.

Certain surgical procedures and conditions result in skin openings which are difficult or not able to be closed with suture. One example is a fasciotomy to alleviate elevated compartment pressure in a swollen limb where the width of the surgical incision is such that the skin edges are too far apart to suture or staple close. In other situations, where there is significant loss of tissue, wound contamination or chronic skin ulcers exist, there is insufficient skin and closure may be difficult or impossible. In these situations, it is desirable to expand the skin from the margins of the opening in the skin by a gradual process of skin stretching and approximation until there is sufficient skin to allow a suture closure.

### SUMMARY OF THE INVENTION

The present invention provides a skin approximator and stretcher that allows an incision to be closed gradually in stepwise or serial fashion over a period of time. The invention allows the separation of the incision halves and forces across the incision to be flexibly adjusted and drawn together during this process, as necessary. Generally the invention provides strips of hook and loop fastener material that may be attached to the skin flanking the incision and one or more spanning strips that draw the two together. The strips are trimmable and packaged in a sterile kit.

Specifically, the present invention provides a kit for incision closure having a closed internally sterile package containing a least two trimmable, sterile flexible incision flanking strips having a first skin contacting side and a second fastener side having hook or loop fasteners and at least one trimmable, sterile flexible spanning element having a skin facing side having hook or loop fasteners releasably mating with the hook or loop fasteners of the incision flanking sides to span the incision contacting each of the incisions flanking sides to draw the incision together.

Thus it is an object of at least one embodiment of the invention to provide a simple disposable skin approximator and stretcher allowing an incision to be drawn together over time.

It is another object of at least one embodiment of the invention to provide a skin approximator and stretcher for an incision that allows the forces and separation of the incision edges to be flexibly adjusted as the incision is drawn together. The use of the hook and loop fasteners allows arbitrarily fine adjustment of separation distances and forces.

The flanking strips are at least 3 centimeters long and from one to four centimeters wide.

It is an object of at least one embodiment of the invention to provide a strip size readily adaptable to abdominal incisions.

The flanking strips provide cutouts on one side space along their length allowing curvature of the strip along a plane of the strip with reduced buckling.

Alternatively, the flanking strips may be cut to size or in segments so as to better fit to the contour of the skin opening.

It is an object of at least one embodiment of the invention to provide cuttable flanking strips to contour to the incisional opening or skin openings such as circular or oval like skin ulcers which can be gradually stretched and approximated over time.

It is another object of at least one embodiment of the invention to provide strips that may accommodate changes in curvature of incision edges as the incision is drawn together over time.

The spanning element may be a set of strips.

It is another object of at least one embodiment of the invention to provide a spanning element that is easily adjustable.

Alternatively, the spanning element may be a panel sized to cover the entire incisional opening.

It is another object of at least one embodiment of the invention to provide a spanning element that provides protection of the incision.

The flanking strips may include printed indicia on one side indicating use of the flanking strips, for example, a side of the flanking strip that is to be away from the skin, and/or a side of the flanking strip that is to be away from an incision edge, and/or a stitch line indicating a preferred location of stitches or staples attaching the flanking strip to the skin.

It is another object of at least one embodiment of the invention to provide a system readily used in the operating room under emergency conditions and/or when a surgeon is gloved.

The printed indicia may indicate a side of the flanking strip that is to be away from an incision edge and the stitch line is near and a side of the flanking strip away from the incision edge as marked.

It is another object of at least one embodiment of the invention to prevent a rolling of the flanking strips under shear forces.

The flanking strips may have a pressure sensitive or other type of adhesive on a side to contact a patient's skin as a replacement to the use of sutures or to be used in conjunction with sutures.

It is another object of at least one embodiment of the invention to stabilize the flanking strips during suturing or to provide an alternative to sutures or staples.

The flanking strips and spanning element may be treated with an antimicrobial material.

It is another object of at least one embodiment of the invention to provide a system that may be used over a period of time with reduced risk of infection.

The present invention contemplates a method of closing an incision having the steps of trimming two sterile, flexible incision flanking strips having a first skin contacting side and, a second fastener side having hook or loop fasteners to an approximate length of the incision and attaching the trimmed flexible incision flanking strips to opposite sides of the incision. The invention further provides steps of joining the attached flexible incision flanking strips with at least one trimmable, sterile flexible lacing element having a skin facing side having hook or loop fasteners releasably mating with the hook or loop fasteners of the incision flanking strips to span the incision contacting each of the incision flanking strips to draw the two together; and as the skin expands releasing the sterile flexible lacing element from the attached flexible incision flanking strips and reattaching the sterile flexible lacing element to the attached flexible incision flanking strips to further stepwise draw the incisional edges or skin opening closer together.

It is another object of at least one embodiment of the invention to provide an improved method of closing an incision particularly of the abdominal skin.

These particular objects and advantages may apply to only some embodiments falling within the claims and thus do not define the scope of the invention.

### BRIEF DESCRIPTION OF THE DRAWINGS

FIG. 1 is a top plan view of an incision being drawn together by the approximator of the present intention using multiple spanning strips;

FIG. 2 is a figure similar to that of FIG. 1 showing an alternative embodiment in which the multiple spanning strips are replaced by a single spanning sheet;

FIG. 3 is an enlarged view of FIG. 1 without the spanning strips showing the labeling of the flanking strips with indicia and showing cutouts allowing curvature of those strips without buckling;

FIG. 4 is a detailed fragmentary view of one flanking strip of FIG. 3 showing outboard location of the sutures indicated by a suture line and showing the labeling in more detail; and

FIG. 5 is a perspective view of a sterile package in which a kit providing the elements of the present invention may be contained.

### DETAILED DESCRIPTION OF THE INVENTION

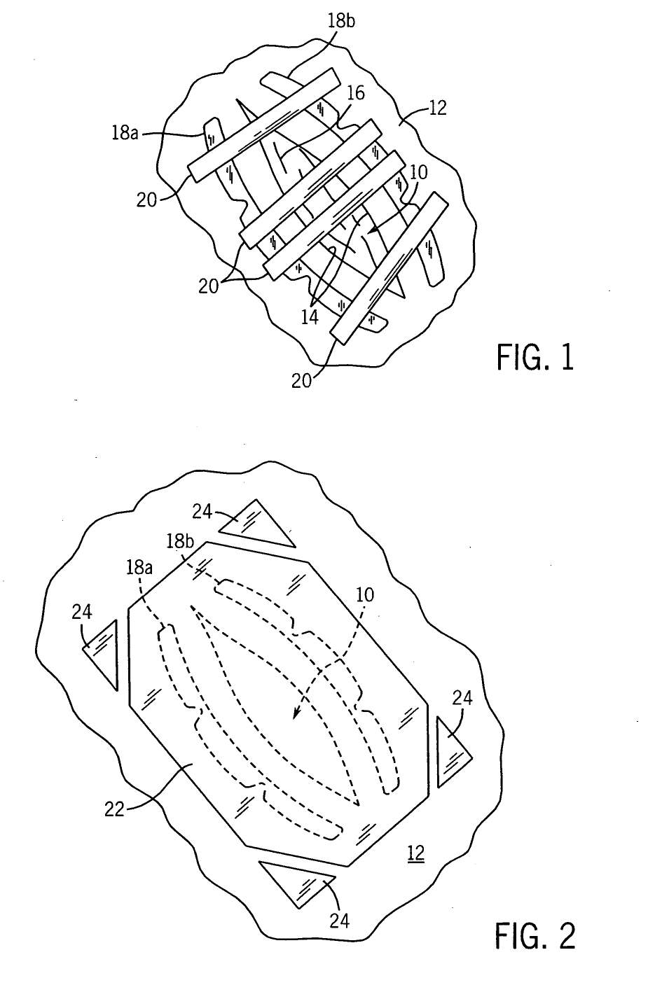

Referring now to FIG. 1, an opening 10 in the skin 12 may have separate opposed skin edges 14 separated as a result of a surgical incision, wound or skin ulcer to expose underlying tissue 16. Generally, the term incision will be used herein to mean any skin opening including those of wounds or ulcers.

In the present invention, flanking strips 18a and 18b may be attached to the skin adjacent to the opening edges 14. Spanning strips 20 may then be used to connect the flanking strips 18a and 18b across the opening 10 over the exposed tissue 16. The use of multiple spanning strips 20 allows the pressure to be exerted across the opening 10 to be adjusted independently from the separation of the edges 14 allowing either even application of force or variations in force to be exerted across those portions of the opening 10 that require it. This ability to tailor the approximating forces and separation distances provides improved incision closure.

Referring now to FIG. 2, in an alternative embodiment, the multiple spanning strips 20 may be replaced with a solid spanning sheet 22. The material of the sheet 22 is trimmable so that corners 24 may be removed if necessary. The solid sheet 22 provides protection of the wound area.

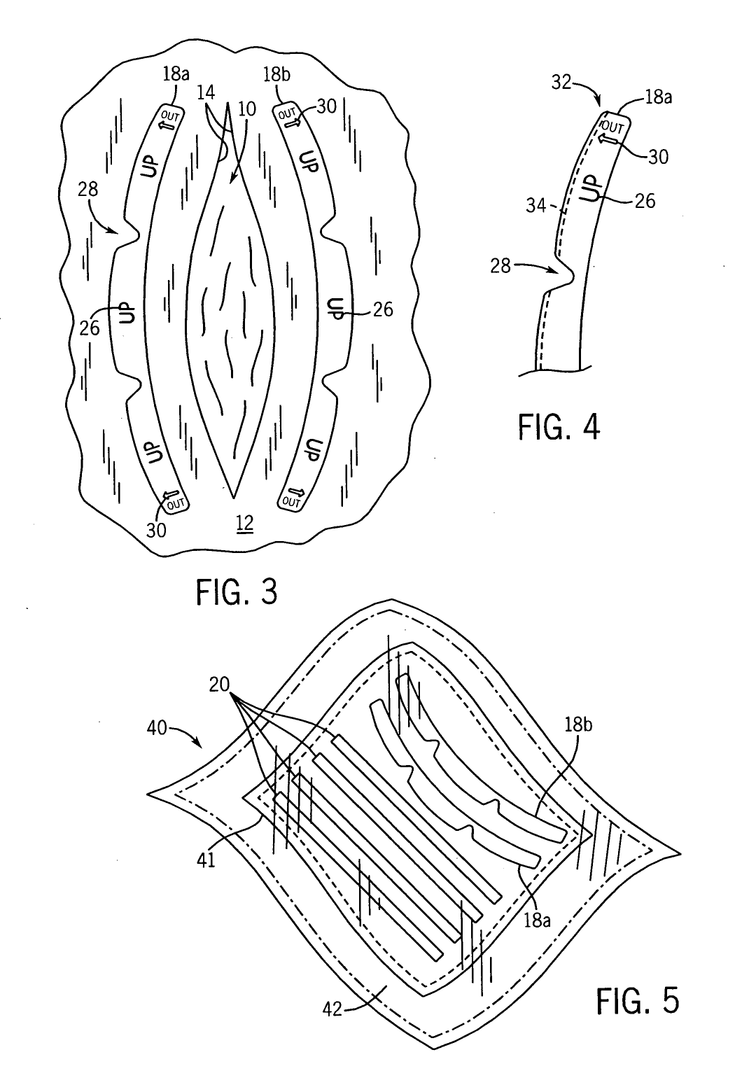

Referring now to FIG. 3, each of the flanking strips 18a and 18b is preferably a loop portion of a hook and loop fastener system such as is manufactured under the trade name Velcro with the loop portion positioned away from the surface of the skin 12 so that the “smooth” side of the material is placed against the skin 12.

The material of the flanking strips 18a and 18b may be devoid of pigment and therefore, it can be difficult to determine which side should be placed upward during surgery when the surgeon's hands are gloved. Accordingly labels 26 may be printed on the upper loop surface indicating that this surface should be facing up.

A second label 30 also printed on the upper surface of the flanking strips 18a and 18b may indicate one edge of the flanking strips 18a and 18b that should be placed away from the incision edges 14. This outer edge may include a series of slits or cutouts 28 passing into the outer edge only partway through the flanking strips 18a and 18b. These cutouts 28 allow the flanking strips 18a and 18b to follow the curvature of the incision edges 14 (by curving within the plane of the skin and flanking strips 18a and 18b), then to straighten as the opening 10 is drawn closed without buckling or rippling. The cutouts 28, nevertheless, keep the flanking strips 18a and 18b as single strips simplifying attachment of flanking strips 18a and 18b at the edges 14 of the opening 10 without the need to deal with many small pieces.

The material of the flanking strips 18a and 18b is trimmable with standard surgical scissors so that the length of the flanking strips 18a and 18b may be adjusted to be similar in length to the length of the incision edges 14. Alternatively, the flanking strips 18a and 18b may be cut into short lengths to piece-wise follow the edges 14 of the opening 10. The flanking strips 18a and 18b may be precut or cut by the physician using scissors or the like from strips or a solid panel for example when a crescent shape is desired.

Referring now to FIG. 4, the flanking strips 18a and 18b may be sutured, stapled, glued or otherwise fixed along their outer edge 32 to so as to prevent rolling of the flanking strips 18a and 18b with force in sheer cause by the spanning strips 20. A dotted line 34 may be printed on this outboard edge to assist the surgeon in properly placing the sutures or staples. The cutouts 28 provide a break in the line of sutures or staples allowing conformal curving of the flanking strips 18a and 18b as the opening 10 closes.

Referring now to FIG. 5, the present invention may be provided as a kit 40 comprising of flanking strips 18a and 18b of length longer than the length of a typical opening 10. Multiple spanning strips 20 may also be provided that may be trimmed to length or cut to make multiple short spanning strips 20 as needed. Or a single spanning sheet 22 may be provided as described above.

Each of strips 20 and 18 may be sterilized and placed within a sterile package 41 for use in the operating room. The sterile package 41 holding a kit 40 may be packaged in yet another sterile package 42 to allow sterile handling of the inside package after opening of the outer package. The sterile packages 41 and 42 may have at least one transparent side to allow visual inspection of the contained strips and of a sterility tag indicating sterility and packaged within the sterile packages.

The material of the flanking strips 18a and 18b, the spanning strips 20, and spanning sheet 22 is detoxified and is biocompatible with respect to skin and tissue contact. Detoxification can be performed by soaking the material in hot distilled water or alcohol to leach out potentially cytotoxic plasticizers or other agents according to methods understood in the art. If not removed, these plasticizers and/or agents make the material unusable for medical tissue contact. In this way, the material may be applied directly to the skin as well as come into contact with tissue 16 eliminating the need for intermediate materials that may add unwanted stiffness to or interference with the interface between the skin and tissue and material.

The material of the flanking strips 18a and 18b, the spanning strips 20, and spanning sheet 22 may be impregnated with silver or other antimicrobials which leaches out over time and serves to protect against or fight infection.

Adhesive may be applied to the back of the flanking strips 18a and 18b or, alternatively, the flanking strips 18a and 18b may have a preapplied pressure sensitive adhesive backing with a peelable protective releasable covering to prevent sticking to other surfaces prior to placement on the skin. This protective covering is removed prior to placement on the skin.

In the preferred embodiment, the flanking strips 18a and 18b are of sufficient width to prevent or minimize blistering. In one embodiment, this may be from 1 to 4 centimeters wide. The width of the flanking strips 18a and 18b are such that shearing forces can be spread over as broad an area as possible when the spanning strips 22 are tensioned or pulled against the flanking strips 18a and 18b during the skin stretching or approximating function as shown in FIG. 1. Suturing of the flanking strips 18a and 18b along the outer edge 34 prevents them from turning over on themselves or peeling away.

The width of both the flanking strips 18a and 18b is determined by the anatomical location where it is used. On the upper and lower extremities the width will be from 1.5 cm to 3.0 cm wide. On the torso or abdominal surface, the width will be approximately 2.0 to 4.0 cm wide. For skin ulcers the strips may be 1.0 to 2.0 cm wide. The length of the flanking strips 18a and 18b will be sufficient to span the outer perimeter of the wound or opening typically at least 3 centimeters, but preferably 35 centimeters or more. For skin ulcers the flanking strips may be 3.0 to 6.0 cm to allow for cutting and conforming to the circular like shape of the ulcer.

The material of the flanking strips 18a and 18b, the spanning strips 20, and spanning sheet 22 may be easily cut to fit by the physician at the time of use. Alternatively, the material of the flanking strips 18a and 18b, the spanning strips 20 and spanning sheet 22 may be precut into convenient lengths.

In an alternative embodiment, the flanking strips 18a and 18b, the spanning strips 20, or spanning sheet 22 can be custom cut to size as desired from one large piece of hook and one large piece of loop material.

As shown in FIG. 2, after the flanking strips 18a and 18b are placed at the edges of the opening 10, the spanning strips 20 are cut and laid in transverse or diagonal fashion across the incision or open wound and adhere to the flanking strips 18a and 18b. Sufficient spanning strips 20 are applied to assure “broad” as opposed to “point” tensioning of the skin along the wound edges. When tension is applied by the spanning strip 20 pulling on the flanking strips 18a and 18b, the skin is gradually stretched. The tension is maintained to allow for gradual skin stretching and approximation. When the maximal skin stretching has been achieved, the flanking strips 18a and 18b are retightened as before causing the skin to stretch and advance still further. The spanning strips 20 are tightened alternatively from one side and then from the other so as to achieve equal pulling of skin edges toward the midline from each side although this is not a requirement for the skin stretching function. When the skin has been stretched to the point that the two edges from either side are close enough and have sufficiently reduced tension, the flanking strips 18a and 18b and spanning strips 20 are removed and the incision edges 14 are sutured, stapled, or otherwise bonded together.

The serial or stepwise tightening of the spanning strips 20 on the flanking strips 18a and 18b take place over time which allows gradual skin and tissue stretching, expansion, and approximation until the skins edges are close enough and sufficiently tension free to allow final closure.

The transverse or diagonal spanning strips 20 may be moved longitudinally along the opening 10 depending on the size and shape of the incision. The spanning strips 20 may be concentrated or grouped in an area where greater skin or tissue stretching, expansion, or approximation is required or desired. In areas where skin tensioning is not required, the spanning strips can be used sparingly, not at all, or left in an untensioned or less tensioned state. Different degrees of tensioning and incision separation may be easily provided by the transverse or diagonal strips. By changing the angle of the diagonal strips, forces with a longitudinal component can be applied to help align the edges of the wound.

When a single sheet 22 is used in lieu of the multiple spanning strips 20, it may be attached at one side of the incision to flanking strip 18a and then pulled gently toward the flanking strip 18b to which it is attached. The corners 24 of the spanning sheet 22 may then be trimmed, or this may be done in advance. The width of the spanning sheet will typically be 10 to 15 centimeters.

It is specifically intended that the present invention not be limited to the embodiments and illustrations contained herein, but include modified forms of those embodiments including portions of the embodiments and combinations of elements of different embodiments as come within the scope of the following claims.

We claim:

1. A kit for incision closure comprising:
a closed internally sterile package;
at least two trimmable, sterile flexible incision flanking strips having a first skin contacting side and a second fastener side having hook or loop fasteners; and
at least one trimmable, sterile flexible spanning element having a skin facing side having hook or loop fasteners releasably mating with the hook or loop fasteners of the incision flanking sides to span the incision contacting each of the incisions flanking sides to draw the incision together.

2. The kit of claim 1 wherein the flanking strip are at least 20 centimeters long and from one to four centimeters wide.

3. The kit of claim 1 wherein the flanking strips provide cutouts on one side spaced along their length allowing curvature of the strip along a plane of the strip with reduced buckling.

4. The kit of claim 1 wherein the flanking strips are loop portions of the hook and loop fastener and the spanning element is a hook portion of the hook and loop fasteners.

5. The kit of claim 1 wherein the spanning element is a set of strips.

6. The kit of claim 1 wherein the spanning element is a panel sized to cover a standard abdominal incision.

7. The kit of claim 1 wherein the flanking strips include printed indicia on one side indicating use of the flanking strips.

8. The kit of claim 7 wherein the printed indicia indicates a side of the flanking strip that is to be away from the skin.

9. The kit of claim 7 wherein the printed indicia indicates a side of the flanking strip that is to be away from an incision edge.

10. The kit of claim 7 wherein the printed indicia is a stitch line indicating a preferred location of stitches or staples attaching the flanking strip to the skin.

11. The kit of claim 10 wherein the printed indicia indicates a side of the flanking strip that is to be away from an incision edge and the stitch line is near a side of the flanking strip away from the incision edge as marked.

12. The kit of claim 1 wherein the flanking strips have a pressure sensitive adhesive on a side to contact a patient's skin.

13. The kit of claim 1 wherein the flanking strips and spanning element are treated with an antimicrobial material.

14. A method of closing an incision comprising the steps of:
(a) trimming two sterile flexible incision flanking strips having a first skin contacting side and a second fastener side having hook or loop fasteners to an approximate length of the incision;
(b) attaching the trimmed flexible incision flanking strips to opposite sides of the incision;
(c) joining the attached flexible incision flanking strips with at least one trimmable, sterile flexible lacing element having a skin facing side having hook or loop fasteners releasably mating with the hook or loop fasteners of the incision flanking strips to span the incision contacting each of the incisions flanking strips to draw the two together; and
(d) periodically releasing the sterile flexible lacing element from the attached flexible incision flanking strips as the skin is stretched and reattaching the sterile flexible lacing element to the attached flexible incision flanking strips to further stretch the skin and stepwise draw the incision closed.

15. The method of claim 14 wherein the step of attaching the trimmed flexible incision flanking strips to opposite sides of the incision attaches the flanking strips at edges away from the incision.

16. The method of claim 14 including the step of trimming the flanking strips to conform to the edges of an incision.
